SEQUENCE LISTING (4 sequences)

SEQ ID NO: 1 - DNA, 4369 bp, RATTUS RATTUS
ttctgccagaattcggcttaactcactatagggctcgaacggcccgcccgggccaggtcgagaagaaatcccacttgattcagatactctccaagattgttaaaggactaacttctccctctttatgcacttacgtctggaagcattcagaaaaggaggaatggaataacaaaggtgactctgtaagcacattcttcaagaaagcccaggcacagtcgaacagcggtttccagaagctgctgatcccatcttgcaaggctcagctcaactcagagccgctgcttcttccaaagtgtacatcttggcggaccttacggaggaaatcggaaagtagagacacgtgaggtcgtgatggagctcgaagaagaccttaacggaagagcagacaagaacttctcaaagatgggcaaaaagagtaaaaaggagaagaaagaaaagaaaccagcggtcagtgtgctcacaatgtttcgctatgcaggttggctggacagattttacatgctgctgggaactctggcggccattatccatggaattgcgctcccacttatgatgctggtctttggagacatgacagatagctttgcaaatgtaggaaacaaccgtagtatgagtttctacaatgctacagacatatatgccaagctggaggacgaaatggccacgtacgcctactattacacgggcaatggtgccggtgtgctcatcgttgcctacatccaggtttcactttggtgcctggcagctgggagacaaatacacaagattaggcagaagtttttccatgccatcatgaatcaggagataggctggtttgacgtgcatgacgttggggagctcaacacccggctcacagatgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaagatattgtcttcatttactgataaggaactccaggcttatgcaaaagctggagcagttgctgaagaagtcttagcagccatcagaactgtgattgcctttggaggacaaaagaaggaacttgaaaggtacaataacaatttggaagaagctaaaaggcttgggataaagaaagctatcacggccaacatttccatgggtgcagcttttctgcttatctatgcatcatatgctctggcattctggtatgggacttccttggtcatctcaaaagaatacactattggacaagtgctcactgtctttttttctgtattaattggagcattcagtgttgggcaggcatctccaaatattgaagccttcgccaatgctagaggagcagcttatgaagtcttcagtataattgataataagcccagtatagacagcttctcaaagagtgggcacaaacccgacaacatacaaggaaatttggaattcaaaaatattcacttcagttacccgtctcgaaaagacgttcagatcttgaagggcctcaacctgaaggtgaagagcgggcagacggtagccctggttggcaacagtggctgtgggaaaagcacaactgtccagctgctgcagaggctctacgaccccatagagggcgaggtcagtatcgacggacaggacatcaggaccatcaatgtgaggtatctgcgggaaatcattggggtggtgagtcaggaacccgtgctgtttgccaccacaattgccgaaaacattcgctatggccgagaaaacgtcaccatggatgagatagagaaagctgtcaaggaagccaatgcctatgatttcatcatgaaactgccccacaaatttgacaccctggttggtgagagaggggcgcagctgagtgggggacagaaacagaggatcgccattgcccgggccctggtccgcaaccccaagatccttttgttggatgaggccacgtcagccttggacacagaaagcgaagccgtggttcaggccgctctggataaggctagagaaggccggaccaccattgtgatagctcaccgcttgtctacagttcgcaatgctgacgtcattgctggttttgatggtggtgtcattgtggagcaaggaaatcatgatgagctcatgagagagaaaggaatttacttcaaacttgtcatgactcagacagcaggaaatgaaattgaattaggaaatgaagcttgtgaatctaaagacggaattgataatgtggacatgtcttcaaaagattcgggatccagtctaataagaagaagatcaactcgcaaaagcatccgtgggccacatgatcaagacggggaacttagcaccaaagaggctctggatgacgacgtacctccagcttccttttggcggatcctgaagttgaattcaactgaatggccttattttgtggttggtgtattttgtgccataataaatggaggcttgcaaccagcattctccataatattttcaaaggttgtaggggtttttacaaaaaatgacacccctgaaatccagcggcagaacagcaacttgttttctttattgttcctgatccttgggatcatctctttcattacgtttttccttcagggcttcacatttggcaaagctggagagatcctcaccaagcgactccgatacatggtcttcaaatccatgctgagacaggacataagctggtttgatgaccctaaaaacaccacaggagcgctgaccaccaggcttgccaatgacgctgctcaagtgaaaggggctacagggtctaggcttgctgttattacccagaacatagcaaatcttgggacaggcatcatcatatccctgatctacggctggcaattgacacttttactcctagcaattgttcccatcattgctatagcaggagtggttgaaatgaaaatgttgtctggacaagcgctgaaagataagaaggaactagaaggttctgggaagatcgctacagaagcaattgaaaactttcgcactgtcgtctctttgactcgggagcagaagtttgaaactatgtatgcccagagcttgcagataccatacagaaatgctttgaagaaagcgcacgtctttgggatcactttctccttcacccaggccatgatgtatttctcctatgctgcttgtttccggtttgatgcctacttggtggcacgagaactcatgacatttgaaaatgttctgttagtattctcagctattgtctttggtgccatggcagtggggcaggtcagttcattcgctcctgactacgcgaaagccaaagtctcggcatcccacatcatcaggatcattgagaaaatccctgagattgacagctacagcacggagggcttgaagcctaatatgttggaaggaaatgtgaaatttaatggagtcatgttcaactatcccacccgacccaacatcccagtgcttcaggggctgagcctagaggtgaagaaagggcagacgctggccctcgtgggcagcagtggctgcgggaagagtacagtggtccagctgcttgagcgcttctatgaccccatggccggaacagtgtttctagatggcaaagaaataaagcaactcaatgtccagtggctccgcgcccacctgggcattgtgtcccaggagcccatcctgtttgactgcagcatcgccgagaacattgcctacggagacaacagccgtgtcgtgtctcataaggagatcgtgaaggcagccaaggaggccaacatccaccagttcatcgactcactgcctgagaaatacaacaccagagtgggagacaaagggactcagctgtcgggcgggcagaagcagcgcatcgccatcgcgcgcgccctcgtcagacagcctcacatcttacttctggatgaagcgacatcagctctggatacggagagtgaaaaggtcgtccaggaagcgctggacaaagccagggaaggccgcacctgcattgtgatcgcgcaccgcctgtccaccatccagaacgcagacttgatcgtggtgattcagaacggccaggtcaaggagcacggcacccaccagcagctgctggcccagaaaggcatctatttctcgatggtcagtgtgcaggctggagcaaagcgctcatgaactgtgaccatgcgagatgttaaatatttttaatgtttgtattaatatatgacacttaatcaaagtcaaaaggaaaacacttactagaatagtcagttatctatttcctgtcacaaaggaaagcatttagtccattttagagtcttcagagactttgtaattaaaagaacaaaaatagatacatcatcaaatggaattc

SEQ ID NO: 2 - protein, 1272 aa, RATTUS RATTUS
MELEEDLNGRADKNFSKMGKKSKKEKKEKKPAVSVLTMFRYAGWLDRFYMLLGTLAAIIHGIALPLMMLVFGDMTDSFANVGNNRSMSFYNATDIYAKLEDEMATYAYYYTGNGAGVLIVAYIQVSLWCLAAGRQIHKIRQKFFHAIMNQEIGWFDVHDVGELNTRLTDDVSKINEGIGDKIGMFFQAMATFFGGFIIGFTRGWKLTLVILAISPVLGLSAGIWAKILSSFTDKELQAYAKAGAVAEEVLAAIRTVIAFGGQKKELERYNNNLEEAKRLGIKKAITANISMGAAFLLIYASYALAFWYGTSLVISKEYTIGQVLTVFFSVLIGAFSVGQASPNIEAFANARGAAYEVFSIIDNKPSIDSFSKSGHKPDNIQGNLEFKNIHFSYPSRKDVQILKGLNLKVKSGQTVALVGNSGCGKSTTVQLLQRLYDPIEGEVSIDGQDIRTINVRYLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQAALDKAREGRTTIVIAHRLSTVRNADVIAGFDGGVIVEQGNHDELMREKGIYFKLVMTQTAGNEIELGNEACESKDGIDNVDMSSKDSGSSLIRRRSTRKSIRGPHDQDGELSTKEALDDDVPPASFWRILKLNSTEWPYFVVGVFCAIINGGLQPAFSIIFSKVVGVFTKNDTPEIQRQNSNLFSLLFLILGIISFITFFLQGFTFGKAGEILTKRLRYMVFKSMLRQDISWFDDPKNTTGALTTRLANDAAQVKGATGSRLAVITQNIANLGTGIIISLIYGWQLTLLLLAIVPIIAIAGVVEMKMLSGQALKDKKELEGSGKIATEAIENFRTVVSLTREQKFETMYAQSLQIPYRNALKKAHVFGITFSFTQAMMYFSYAACFRFDAYLVARELMTFENVLLVFSAIVFGAMAVGQVSSFAPDYAKAKVSASHIIRIIEKIPEIDSYSTEGLKPNMLEGNVKFNGVMFNYPTRPNIPVLQGLSLEVKKGQTLALVGSSGCGKSTVVQLLERFYDPMAGTVFLDGKEIKQLNVQWLRAHLGIVSQEPILFDCSIAENIAYGDNSRVVSHKEIVKAAKEANIHQFIDSLPEKYNTRVGDKGTQLSGGQKQRIAIARALVRQPHILLLDEATSALDTESEKVVQEALDKAREGRTCIVIAHRLSTIQNADLIVVIQNGQVKEHGTHQQLLAQKGIYFSMVSVQAGAKRS

SEQ ID NO: 3 - DNA, 4425 bp, RATTUS RATTUS
ttctgccagaattcggcttaactcactatagggctcgaacggcccgcccgggccaggtcgagaagaaatcccacttgattcagatactctccaagattgttaaaggactaacttctccctctttatgcacttacgtctggaagcattcagaaaaggaggaatggaataacaaaggtgactctgtaagcacattcttcaagaaagcccaggcacagtcgaacagcggtttccagaagctgctgatcccatcttgcaaggctcagctcaactcagagccgctgcttcttccaaagtgtacatcttggcggaccttacggaggaaatcggaaagtagagacacgtgaggtcgtgatggagctcgaagaagaccttaacggaagagcagacaagaacttctcaaagatgggcaaaaagagtaaaaaggagaagaaagaaaagaaaccagcggtcagtgtgctcacaatgtttcgctatgcaggttggctggacagattttacatgctgctgggaactctggcggccattatccatggaattgcgctcccacttatgatgctggtctttggagacatgacagatagctttgcaaatgtaggaaacaaccgtagtatgagtttctacaatgctacagacatatatgccaagctggaggacgaaatgaccacgtacgcctactattacacgggcattggtgccggtgtgctcatcgttgcctacatccaggtttccacttggtgcctggcagctgggagacaaatacacaagattaggcagaagtttttccatgccatcatgaatcaggagataggctggtttgacgtgcatgacgttggggagctcaacacccggctcacagatgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaagatattgtcttcatttactgataaggaactccaggcttatgcaaaagctggagcagttgctgaagaagtcttagcagccatcagaactgtgattgcctttggaggacaaaagaaggaacttgaaaggtacaataacaatttggaagaagctaaaaggcttgggataaagaaagctatcacggccaacatttccatgggtgcagcttttctgcttatctatgcatcatatgctctggcattctggtatgggacttccttggtcatctcaaaagaatacactattggacaagtgctcactgtctttttttctgtattaattggagcattcagtgttgggcaggcatctccaaatattgaagccttcgccaatgctagaggagcagcttatgaagtcttcagtataattgataataagcccagtatagacagcttctcaaagagtgggcacaaacccgacaacatacaaggaaatttggaattcaaaaatattcacttcagttacccgtctcgaaaagacgttcagatcttgaagggcctcaacctgaaggtgaagagcgggcagacggtagccctggttggcaacagtggctgtgggaaaagcacaactgtccagctgctgcagaggctctacgaccccatagagggcgaggtcagtatcgacggacaggacatcaggaccatcaatgtgaggtatctgcgggaaatcattggggtggtgagtcaggaacccgtgctgtttgccaccacaattgccgaaaacattcgctatggccgagaaaacgtcaccatggatgagatagagaaagctgtcaaggaagccaatgcctatgatttcatcatgaaactgccccacaaatttgacaccctggttggtgagagaggggcgcagctgagtgggggacagaaacagaggatcgccattgcccgggccctggtccgcaaccccaagatccttttgttggatgaggccacgtcagccttggacacagaaagcgaagccgtggttcaggccgctctggataaggctagagaaggccggaccaccattgtgatagctcaccgcttgtctacagttcgcaatgctgaygtcattgctggttttgatggtggtgtcattgtggagcaaggaaatcatgatgagctcatgagagagaaaggaatttacttcaaacttgtcatgactcagacagcaggaaatgaaattgaattaggaaatgaagcttgtgaatctaaagayggaattgataatgtggacatgtcttcaaaagattcrggatccagtctaataagaagaagatcaactcgcaaaagcatccgtgggccacatgatcaagacggggaacttagcaccaaagaggctctggatgacgacgtacctccagcttccttttggcggatcctgaagttgaattcaactgaatggccttattttgtggttggtgtattttgtgccataataaatggaggcttgcaaccagcattctccataatattttcaaaggttgtaggggtttttacaaaaaatgacacccctgaaatccagcggcagaacagcaacttgttttctttattgttcctgatccttgggatcatctctttcattacgtttttccttcagggcttcacatttggcaaagctggagagatcctcaccaagcgactccgatacatggtcttcaaatccatgctgagacaggacataagctggtttgatgaccctaaaaacaccacaggagcgctgaccaccaggcttgccaatgacgctgctcaagtgaaaggggctacagggtctaggcttgctgttattacccagaacatagcaaatcttgggacaggcatcatcatatccctgatctacggctggcaattgacacttttactcctagcaattgttcccatcattgctatagcaggagtggttgaaatgaaaatgttgtctggacaagcgctgaaagataagaaggaactagaaggttctgggaagatcgctacagaagcaattgaaaactttcgcactgtcgtctctttgactcgggagcagaagtttgaaactatgtatgcccagagcttgcagataccatacagaaatgctttgaagaaagcgcacgtctttgggatcactttctccttcacccaggccatgatgtatttctcctatgctgcttgtttccggtttgatgcctacttggtggcacgagaactcatgacatttgaaaatgttctgttagtattctcagctattgtctttggtgccatggcagtggggcaggtcagttcattcgctcctgactacgcgaaagccaaagtctcggcatcccacatcatcaggatcattgagaaaatccctgagattgacagctacagcacggagggcttgaagcctaatatgttggaaggaaatgtgaaatttaatggagtcatgttcaactatcccacccgacccaacatcccagtgcttcaggggctgagcctagaggtgaagaaagggcagacgctggccctcgtgggcagcagtggctgcgggaagagtacagtggtccagctgcttgagcgcttctatgaccccatggccggaacagtgtttctagatggcaaagaaataaagcaactcaatgtccagtggctccgcgcccacctgggcattgtgtcccaggagcccatcctgtttgactgcagcatcgccgagaacattgcctacggagacaacagccgtgtcgtgtctcataaggagatcgtgaaggcagccaaggaggccaacatccaccagttcatcgactcactgcctgagaaatacaacaccagagtgggagacaaagggactcagctgtcgggcgggcagaagcagcgcatcgccatcgcgcgcgccctcgtcagacagcctcacatcttacttctggatgaagcgacatcagctctggatacggagagtgaaaaggtcgtccaggaagcgctggacaaagccagggaaggccgcacctgcattgtgatcgcgcaccgcctgtccaccatccagaacgcagacttgatcgtggtgattcagaacggccaggtcaaggagcacggcacccaccagcagctgctggcccagaaaggcatctatttctcgatggtcagtgtgcaggctggagcaaagcgctcatgaactgtgaccatgcgagatgttaaatatttttaatgtttgtattaatatatgacacttaatcaaagtcaaaaggaaaacacttactagaatagtcagttatctatttcctgtcacaaaggaaagcatttagtccattttagagtcttcagagactttgtaattaaaagaacaaaaatagatacatcatcaaatggaattcctgcagcccgggggatccactagttctagagcggccgccaccgcggtggagctcca

SEQ ID NO: 4 - protein, 1272 aa, RATTUS RATTUS
MELEEDLNGRADKNFSKMGKKSKKEKKEKKPAVSVLTMFRYAGWLDRFYMLLGTLAAIIHGIALPLMMLVFGDMTDSFANVGNNRSMSFYNATDIYAKLEDEMTTYAYYYTGIGAGVLIVAYIQVSTWCLAAGRQIHKIRQKFFHAIMNQEIGWFDVHDVGELNTRLTDDVSKINEGIGDKIGMFFQAMATFFGGFIIGFTRGWKLTLVILAISPVLGLSAGIWAKILSSFTDKELQAYAKAGAVAEEVLAAIRTVIAFGGQKKELERYNNNLEEAKRLGIKKAITANISMGAAFLLIYASYALAFWYGTSLVISKEYTIGQVLTVFFSVLIGAFSVGQASPNIEAFANARGAAYEVFSIIDNKPSIDSFSKSGHKPDNIQGNLEFKNIHFSYPSRKDVQILKGLNLKVKSGQTVALVGNSGCGKSTTVQLLQRLYDPIEGEVSIDGQDIRTINVRYLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQAALDKAREGRTTIVIAHRLSTVRNADVIAGFDGGVIVEQGNHDELMREKGIYFKLVMTQTAGNEIELGNEACESKDGIDNVDMSSKDSGSSLIRRRSTRKSIRGPHDQDGELSTKEALDDDVPPASFWRILKLNSTEWPYFVVGVFCAIINGGLQPAFSIIFSKVVGVFTKNDTPEIQRQNSNLFSLLFLILGIISFITFFLQGFTFGKAGEILTKRLRYMVFKSMLRQDISWFDDPKNTTGALTTRLANDAAQVKGATGSRLAVITQNIANLGTGIIISLIYGWQLTLLLLAIVPIIAIAGVVEMKMLSGQALKDKKELEGSGKIATEAIENFRTVVSLTREQKFETMYAQSLQIPYRNALKKAHVFGITFSFTQAMMYFSYAACFRFDAYLVARELMTFENVLLVFSAIVFGAMAVGQVSSFAPDYAKAKVSASHIIRIIEKIPEIDSYSTEGLKPNMLEGNVKFNGVMFNYPTRPNIPVLQGLSLEVKKGQTLALVGSSGCGKSTVVQLLERFYDPMAGTVFLDGKEIKQLNVQWLRAHLGIVSQEPILFDCSIAENIAYGDNSRVVSHKEIVKAAKEANIHQFIDSLPEKYNTRVGDKGTQLSGGQKQRIAIARALVRQPHILLLDEATSALDTESEKVVQEALDKAREGRTCIVIAHRLSTIQNADLIVVIQNGQVKEHGTHQQLLAQKGIYFSMVSVQAGAKRS

(19) United States
(12) Patent Application Publication
(10) Pub. No.: US 2002/0055128 A1
(43) Pub. Date: May 9, 2002

(54) Polynucleotide and polypeptide sequences encoding rat mdr1a and screening methods thereof

(76) Inventors: Kimberly Anne Brun, Harleysville, PA (US); Richard James Chenery, Hitchin (GB); Harma Ellens, Norristown, PA (US); John Anthony Feild, Wayne, PA (US); Lin Yue, Phoenixville, PA (US)

Correspondence Address: Ratner & Prestia, P.O. Box 980, Valley Forge, PA, 19482-0980, US

(21) Appl. No.: 09/769,097

(22) Filed: Jan. 25, 2001

Related U.S. Application Data
(63) Continuation of application No. 09/208,809, filed on Dec. 9, 1998, now abandoned.
Application No. 09/208,809 is a continuation-in-part of application No. 09/156,800, filed on Sep. 17, 1998, now abandoned.

(51) Int. Cl. G01N 33/567; C07H 21/04; C12N 9/00; C12N 5/06; C12P 21/02

(52) U.S. Cl. 435/7.21; 435/183; 435/69.1; 435/325; 435/320.1; 536/23.2

(57) ABSTRACT

Rat mdr1a polypeptides and polynucleotides and method for producing such polypeptides by recombinant techniques are disclosed. Also disclosed are methods for screening for compounds which either agonize or antagonize rat mdr1a. Further disclosed is method for performing a selection screen, whereby compounds are discovered that neither agonize nor antagonize rat mdr1a.

### CROSS-REFERENCE TO RELATED APPLICATIONS

This application is a continuation-in-part of and claims priority to U.S. application Ser. No. 09/156,800, which was filed on Sep. 17, 1998, the contents of which are herein incorporated by reference in their entirety.

### FIELD OF THE INVENTION

This invention relates to newly identified polypeptides and polynucleotides encoding such polypeptides, to their use in identifying compounds which may be agonists, antagonists and/or inhibitors, and to production of such polypeptides and polynucleotides.

### BACKGROUND OF THE INVENTION

The drug discovery process is currently undergoing a fundamental revolution as it embraces ‘functional genomics’, that is, high throughput genome- or gene-based biology. This approach is rapidly superceding earlier approaches based on ‘positional cloning’. A phenotype, that is a biological function or genetic disease, would be identified and this would then be tracked back to the responsible gene, based on its genetic map position.

Functional genomics relies heavily on the various tools of bioinformatics to identify gene sequences of potential interest from the many molecular biology databases now available. There is a continuing need to identify and characterise further genes and their related polypeptides/proteins, as targets for drug discovery.

Multi-specific drug transporters are present in cells having a barrier function such as intestinal epithelial and brain microvessel endothelial cells. Other tissues, for example, liver and kidney, also contain multi-specific transporters that can mediate the excretion of drugs and metabolites. Recently, it has been recognized that transporters encoded by genes of MDR (multidrug resistance protein) family proteins contribute to poor intestinal absorption and brain penetration of drugs. Information gained from using multi-specific transporters such as the rat mdr1a gene product in cell based, membrane based, binding or other assays could enhance drug formulation, selection of formulation excipients, and compound design.

### SUMMARY OF THE INVENTION

The present invention relates to rat mdr1a, in particular rat mdr1a polypeptides and rat mdr1a polynucleotides, recombinant materials and methods for their production. In another aspect, the invention relates to methods for identifying agonists and antagonists/inhibitors of the rat mdr1a gene, as well as compounds that neither agonize nor antagonize the rat mdr1a gene. This invention further relates to the generation of in vitro and in vivo comparison data to predict oral absorption and pharmacokinetics in man. Such a comparison of data will enable selection of drugs with optimal pharmacokinetics in man, i.e., good oral bioavailability, brain penetration, plasma half life, and minimum drug interaction.

The present invention further relates to methods for creating transgenic animals and knock-out animals. Furthermore, this invention relates to transgenic and knock-out animals obtained by using these methods. Such animal models are expected to provide valuable insight into the potential pharmacological and toxicological effects in humans of compounds that are discovered by the aforementioned screening methods. An understanding of how the rat mdr1a gene functions in these animal models is expected to provide an insight into treating and preventing human diseases including, but not limited to, cancer, inflammation, cardiovascular disease, central nervous system disorders, auto-immune and kidney disease.

### DESCRIPTION OF THE INVENTION

In a first aspect, the present invention relates to rat mdr1a polypeptides. Such peptides include isolated polypetides comprising an amino acid sequence which has at least a 95% identity to that of SEQ ID NO:2 over the entire length of SEQ ID NO:2. Such polypeptides include those comprising the amino acid of SEQ ID NO:2.

Further peptides of the present invention include isolated polypeptides in which the amino acid sequence has at least a 95% identity, to the amino acid sequence of SEQ ID NO:2 over the entire length of SEQ ID NO:2. Such polypeptides include the polypeptide of SEQ ID NO: 2.

Further peptides of the present invention include isolated polypeptides encoded by a polynucleotide comprising the sequence contained in SEQ ID NO:1.

Polypeptides of the present invention are believed to be members of the multi-specific drug transporters family of polypeptides. They are therefore of interest because they can be used to establish assays to predict oral absorbtion and pharmacokinetics and thus enhance compound and formulation design. These properties are hereinafter referred to as “rat mdr1a activity” or “rat mdr1a polypeptide activity” or “biological activity of mdr1a.” Preferably, a polypeptide of the present invention exhibits at least one biological activity of rat mdr1a.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes variants of the aforementioned polypetides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

In a further aspect, the present invention relates to rat mdr1a polynucleotides. Such polynucleotides include isolated polynucleotides comprising a nucleotide sequence encoding a polypeptide which has at least a 90% identity to the amino acid sequence of SEQ ID NO:2, over the entire length of SEQ ID NO:2. Such polynucleotides include a polynucleotide comprising the nucleotide sequence contained in SEQ ID NO:1 encoding the polypeptide of SEQ ID NO:2.

Further polynucleotides of the present invention include isolated polynucleotides comprising a nucleotide sequence that has at least a 90% identity to a nucleotide sequence encoding a polypeptide of SEQ ID NO:2, over the entire coding region.

Further, polynucleotides of the present invention include isolated polynucleotides comprising a nucleotide sequence which has at least a 90% identity to SEQ ID NO:1 over the entire length of SEQ ID NO:1. Such polynucleotides include a polynucleotide comprising the polynucleotide of SEQ ID NO:1 as well as the polynucleotide of SEQ ID NO:1.

The invention also provides polynucleotides which are complementary to all the above described polynucleotides. The nucleotide sequence of SEQ ID NO:1 shows homology (17% identity over the entire length of SEQ ID NO 1, 99% identity over a 763 nucleotide fragment) with the partial sequence of rat mdr1a (763 nucleiotide) gene, which is published by Teeter, L. D., et al., Mol. Carcinog. 1993, 8:67-73). The nucleotide sequence of SEQ ID NO:1 is a full length cDNA sequence and comprises a polypeptide encoding sequence (nucleotide 352 to 4170) encoding a polypeptide of 1272 amino acids, the polypeptide of SEQ ID NO:2. The nucleotide sequence encoding the polypeptide of SEQ ID NO:2 may be identical to the polypeptide encoding sequence contained in SEQ ID NO:1 or it may be a sequence other than the one contained in SEQ ID NO:1, which, as a result of the redundancy (degeneracy) of the genetic code, also encodes the polypeptide of SEQ ID NO:2. The polypeptide of the SEQ ID NO:2 is structurally related to other proteins of the multi-specific drug transporters family, having homology and/or structural similarity with human MDR1 (86% identity over the entire coding region, for human MDR1 sequence, please see references: Chen, C. J., et al., Cell, 1986, 47:381-389; Ueda, K. et al., J. Biol. Chem, 1987, 262:505-508), mouse mdr1a (94% identity over entire coding region, for mouse mdr1a sequence, please see reference:Hsu, S.I et al, Mol Cell Biol., 1990, 10:3596-3606), rat mdr1b (84% identity over the entire coding region, for rat mdr1b sequence please see reference: Silverman, J. A. et al., Gene, 1991, 106:229-236).

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one mdr1a activity.

The present invention also relates to partial or other polynucleotide and polypeptide sequences which were first identified prior to the determination of the corresponding full length sequences of SEQ ID NO:1 and SEQ ID NO:2.

Accordingly, in a further aspect, the present invention provides for an isolated polynucleotide comprising:

(a) a nucleotide sequence which has at least 70% identity, preferably at least 80% identity, more preferably at least 90% identity, yet more preferably at least 95% identity, even more preferably at least 97-99% identity to SEQ ID NO:3 over the entire length of SEQ ID NO:3;

(b) a nucleotide sequence which has at least 70% identity, preferably at least 80% identity, more preferably at least 90% identity, yet more preferably at least 95% identity, even more preferably at least 97-99% identity, to SEQ ID NO:3 over the entire length of SEQ ID NO:3;

(c) the polynucleotide of SEQ ID NO:3; or

(d) a nucleotide sequence encoding a polypeptide which has at least 70% identity, preferably at least 80% identity, more preferably at least 90% identity, yet more preferably at least 95% identity, even more preferably at least 97-99% identity, to the amino acid sequence of SEQ ID NO: 4, over the entire length of SEQ ID NO:4; as well as the polynucleotide of SEQ ID NO:3.

The present invention further provides for a polypeptide which:

(a) comprises an amino acid sequence which has at least 70% identity, preferably at least 80% identity, more preferably at least 90% identity, yet more preferably at least 95% identity, most preferably at least 97-99% identity, to that of SEQ ID NO:4 over the entire length of SEQ ID NO: 4;

(b) has an amino acid sequence which is at least 70% identity, preferably at least 80% identity, more preferably at least 90% identity, yet more preferably at least 95% identity, most preferably at least 97-99% identity, to the amino acid sequence of SEQ ID NO:4 over the entire length of SEQ ID NO:4;

(c) comprises the amino acid of SEQ ID NO:4; and

(d) is the polypeptide of SEQ ID NO:4; as well as polypeptides encoded by a polynucleotide comprising the sequence contained in SEQ ID NO:3.

The nucleotide sequence of SEQ ID NO:3 and the peptide sequence encoded thereby are derived from EST (Expressed Sequence Tag) sequences. It is recognised by those skilled in the art that there will inevitably be some nucleotide sequence reading errors in EST sequences (see Adams, M. D. et al, Nature 377 (supp) 3, 1995). Accordingly, the nucleotide sequence of SEQ ID NO: 3 and the peptide sequence encoded therefrom are therefore subject to the same inherent limitations in sequence accuracy. Furthermore, the peptide sequence encoded by SEQ ID NO:3 comprises a region of identity or close homology and/or close structural similarity (for example a conservative amino acid difference) with the closest homologous or structurally similar protein.

Polynucleotides of the present invention may be obtained, using standard cloning and screening techniques, from a cDNA library derived from mRNA in cells of tissues including, but not limised to, rat kidney, small intestine, brain, liver, adrenal, ovary, testes, muscle, and lung using the expressed sequence tag (EST) analysis (Adams, M. D., et al. Science (1991) 252:1651-1656; Adams, M. D. et al, Nature (1992) 355:632-634; Adams, M. D., et al, Nature (1995) 377 Supp.: 3-174). Polynucleotides of the invention can also be obtained from sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself; or the coding sequence for the mature polypeptide in reading frame with other coding sequences, such as those encoding a leader or secretory sequence, a pre-, or pro- or prepro- protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz, et al, Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Further embodiments of the present invention include polynucleotides encoding polypeptide variants which comprise the amino acid sequence of SEQ ID NO:2 and in which several, for instance from 5 to 10, 1 to 5, 1 to 3, 1 to 2 or 1, amino acid residues are substituted, deleted or added, in any combination.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than rat) that have a high sequence similarity to SEQ ID NO:1. Typically these nucleotide sequences are 90% identical to that of the referent. The probes or primers will generally comprise at least 15 nucleotides, preferably, at least 30 nucleotides and may have at least 50 nucleotides. Particularly preferred probes will have between 30 and 50 nucleotides.

A polynucleotide encoding a polypeptide of the present invention, including homologs and orthologs from species other than rat, may be obtained by a process which comprises the steps of screening an appropriate library under stringent hybridization conditions with a labeled probe having the sequence of SEQ ID NO:1 or a fragment thereof; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 microgram/ml denatured, sheared salmon sperm DNA; followed by washing the filters in 0.1×SSC at about 65° C. Thus the present invention also includes polynucleotides obtainable by screening an appropriate library under stringent hybridization conditions with a labeled probe having the sequence of SEQ ID NO:1 or a fragment thereof.

The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low ‘processivity’ (a measure of the ability of the enzyme to remain attached to the template during the polymerisation reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman, et al., Proc. Natl. Acad. Sci., USA 85, 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analysed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems which comprise a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis, et al., BASIC METHODS IN MOLECULAR BIOLOGY (1986) and Sambrook,et al., MOLECULAR CLONING: A LABORATORY MANUAL, 2nd Ed., Cold Spring Harbor Laboratory Press, Cold Spring Harbor, N.Y. (1989). Preferred such methods include, for instance, calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293, LLC-PK, MDCK, and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook, et al., MOLECULAR CLONING, A LABORATORY MANUAL (supra).

If a polypeptide of the present invention is to be expressed for use in screening assays, it is generally preferred that the polypeptide be produced at the surface of the cell. In this event, the cells may be harvested prior to use in the screening assay.

The polypeptides of the present invention, as well as cell membranes that express the polypeptides of the invention, can be recovered and purified from recombinant cell cultures by well-known methods including ammonium sulfate or ethanol precipitation, acid extraction, differential centrifugation, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

The rat mdr1a gene products can be expressed in transgenic animals. Animals of any species, including, but not limited to, mice, rats, rabbits, guinea pigs, pigs, micro-pigs, goats, and non-human primates, e.g., baboons, monkeys, chimpanzees, may be used to generate mdr1a transgenic animals.

This invention further relates to a method of producing transgenic animals, preferably rats, over-expressing mdr1a, which method comprises the introduction of several copies of a segment comprising at least the polynucleotide sequence of SEQ ID NO:1 with a suitable promotor into the cells of a rat embryo at an early stage.

This invention also relates to transgenic animals, characterized in that they are obtained by the method of making transgenic rats, as defined above.

Any technique known in the art may be used to introduce the rat mdr1a transgene into animals to produce the founder line of animals. Such techniques include, but are not limited to: pronuclear microinjection (U.S. Pat. No. 4,873,191); retrovirus mediated gene transfer into germ lines (Van der Putten, et al., Proc. Natl. Acad. Sci., USA 82: 6148-6152 (1985); gene targeting in embryonic stem cells (Thompson, et al., Cell 56: 313-321 (1989); electrapolation of embryos (Lo, Mol. Cell Biol. 3: 1803-1814 (1983); and sperm-mediated gene transfer (Lavitrano, et al., Cell 57: 717-723 (1989); etc. For a review of such techniques, see Gordon, Intl. Rev. Cytol. 115: 171-229 (1989).

A further aspect of the present invention involves gene targeting by homologous recombination in embryonic stem cells to produce a transgenic animal with a mutation in the mdr1a gene (“knock-out” mutation). In such so-called “knock-out” animals, there is inactivation of the mdr1a gene or altered gene expression, such that the animals can be useful to study the function of the mdr1a gene, the pharmacological and toxicological properties of a wide range of drugs, thus providing animal models for human diseases, as well as for use in pharmacological, toxicological studies, which are otherwise not readily available through spontaneous, chemical or irradiation mutagenesis.

This invention further relates to a method of producing “knock-out” animals, preferably rats, no longer expressing mdr1a, characterized in that:

a suitable mutation is produced in the polynucleotide sequence of SEQ ID NO:1 conforming to the invention which inhibits the expression of the gene which encodes the rat mdr1a;

the said modified SEQ ID NO:1 is introduced into a segment of rat genomic DNA, combined with an appropriate marker, so as to obtain a labelled sequence containing the modified sequence of SEQ ID NO:1;

the said modified SEQ ID NO:1 is integrated in vitro into the stem cells of rat embryo germ lines; then

the said stem cells are reinjected into a rat; and after homozygous recombination,

homozygous recombinant rats are obtained at the F2 generation which are recognizable by the presence of the marker.

Various methods for producing mutations are contemplated and well known in the art. Preferred is a method where a mutation is generated in a rat mdr1a allele by the introduction of a DNA construct containing DNA of a gene encoding rat mdr1a and the mutation accommodated therein. The mutation is targeted to the allele by way of the DNA construct. The DNA of the gene encoding rat mdr1a contained by the construct may be foreign to the species of which the recipient is a member, as in exogenous DNA, or native to the species and foreign only to the individual recipient, as in isogenous DNA, or a mixture of both. The mutation may constitute an insertion, deletion, substitution, or combination thereof. The DNA construct can be introduced by, for example, calcium-phosphate DNA co-precipitation. It is preferred that a mutation be introduced into a electroporation, microinjection, virus infection, ligand-DNA conjugation, virus-ligand-DNA conjugation, and liposomes.

Another embodiment of the instant invention is “knock-out” animals, preferably rats, characterized in that they are obtained by the method of producing recombinant rats as defined above.

The transgenic and “knock-out” animals as defined above are a particularly advantageous model, from a physiological point of view, for studying multi-specific drug transporters. Such animals will be valuable tools to study the function of the rat mdr1a gene. Moreover, such animal models are expected to provide information about potential toxicological effects in humans of any compounds that are discovered by the aforementioned screening methods. An understanding of how the rat mdr1a gene functions in these animal models is expected to provide an insight into treating and preventing human diseases including, but not limited to, cancer, inflammation, cardiovascular disease, central nervous system disorders, auto-immune and kidney disease.

Polypeptides of the present invention are responsible for many biological functions, including many disease states, in particular the diseases hereinbefore mentioned. It is therefore desirous to devise screening methods to identify compounds which stimulate or which inhibit the function of the polypeptide. Accordingly, in a further aspect, the present invention provides for a method of screening compounds to identify those which stimulate or which inhibit the function of the polypeptide. In general, agonists or antagonists may be employed for therapeutic and prophylactic purposes for such diseases as hereinbefore mentioned. Compounds may be identified from a variety of sources, for example, cells, cell-free preparations, chemical libraries, and natural product mixtures. Such agonists, antagonists or inhibitors so-identified may be natural or modified substrates, ligands, receptors, enzymes, etc., as the case may be, of the polypeptide; or may be structural or functional mimetics thereof (see Coligan, et al., CURRENT PROTOCOLS IN IMMUNOLOGY 1(2): Chapter5 (1991)).

The screening method may simply measure the binding of a candidate compound to the polypeptide, or to cells or membranes bearing the polypeptide, or a fusion protein thereof by means of a label directly or indirectly associated with the candidate compound. Alternatively, the screening method may involve competition with a labeled competitor. Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Constitutively active polypeptides may be employed in screening methods for inverse agonists or inhibitors, in the absence of an agonist or inhibitor, by testing whether the candidate compound results in inhibition of activation of the polypeptide. Further, the screening methods may simply comprise the steps of mixing a candidate compound with a solution containing a polypeptide of the present invention, to form a mixture, measuring rat mdr1a activity in the mixture, and comparing the rat mdr1a activity of the mixture to a standard. Fusion proteins, such as those made from Fc portion and rat mdr1a polypeptide, as hereinbefore described, can also be used for high-throughput screening assays to identify antagonists for the polypeptide of the present invention (see D. Bennett, et al., J. Mol. Recognition, 8:52-58 (1995); and K. Johanson, et al, J. Biol. Chem., 270(16):9459-9471 (1995)).

Cell lines can be established which are transfected with the recombinant gene and expressing the transporter gene product. Whole cell or membrane assays can be developed which will evaluate and quantitate the interactions of drugs and test compounds with the transporter.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented.

Thus, in another aspect, the present invention relates to a screening kit for identifying agonists, antagonists, ligands, substrates, enzymes, etc. for polypeptides of the present invention; or compounds which decrease or enhance the production of such polypeptides, which comprises:

(a) a polypeptide of the present invention;

(b) a recombinant cell expressing a polypeptide of the present invention;

(c) a cell membrane expressing a polypeptide of the present invention; or

(d) antibody to a polypeptide of the present invention; which polypeptide is preferably that of SEQ ID NO:2.

It will be appreciated that in any such kit, (a), (b), (c) or (d) may comprise a substantial component.

It will be readily appreciated by the skilled artisan that a polypeptide of the present invention may also be used in a method for the structure-based design of an agonist, antagonist or inhibitor of the polypeptide, by:

(a) determining in the first instance the three-dimensional structure of the polypeptide;

(b) deducing the three-dimensional structure for the likely reactive or binding site(s) of an agonist, antagonist or inhibitor;

(c) synthesizing candidate compounds that are predicted to bind to or react with the deduced binding or reactive site; and

(d) testing whether the candidate compounds are indeed agonists, antagonists or inhibitors.

It will be further appreciated that this will normally be an interactive process.

Multi-specific drug transporters, such as mdr1, are present in cells having a barrier function, such as intestinal epithelial cells, brain microvessel endothelial cells, kidney epithelial cells, and liver hepatocytes. It was recently recognized that these transporters contribute to poor intestinal absorption, poor penetration into the brain, rapid plasma clearance and variability, as well as drug interactions.

In a preferred embodiment, the present invention relates to the use of rat mdr1a polypeptides, polynucleotides, and recombinant materials thereof in selection screens to identify compounds which are not agonists or antagonist/inhibitors of rat mdr1a. The data from such a selection screen and a similar screen for human mdr1 is expected to provide in vitro and in vivo comparisons to predict oral absorption, pharmacokinetics in humans. The ability to make such a comparison of data will enhance formulation design through the identification of compounds with optimal development characteristics, i.e., high oral bioavailability, UID (once a day) dosing, reduced drug interactions, reduced variability, and reduced food effects, specifically to avoid interacting with human mdr- 1.

The following definitions are provided to facilitate understanding of certain terms used frequently hereinbefore.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Knock-out” refers to partial or complete suppression of the expression of at least a portion of a protein encoded by an endogenous DNA sequence in a cell.

“Transgenic animal” refers to an animal to which foreign DNA has been introduced while the animal is still in its embryonic stage. In most cases, the transgenic approach aims at specific modifications of the genome, e.g., by introducing whole transcriptional units into the genome, or by inactivating pre-existing cellular genes. The targeted character of these procedures sets transgenic technologies apart from experimental methods in which random mutations are conferred to the germline, such as administration of chemical mutagens or treatment with ionizing solution.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS-STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth Enzymol (1990) 182:626-646 and Rattan, et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J Molec Biol (1990) 215:403).

As an illustration, by a polynucleotide having a nucleotide sequence having at least, for example, 95% “identity” to a reference nucleotide sequence of SEQ ID NO:1 is intended that the nucleotide sequence of the polynucleotide is identical to the reference sequence except that the polynucleotide sequence may include up to five point mutations per each 100 nucleotides of the reference nucleotide sequence of SEQ ID NO:1. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% identity to a reference amino acid sequence of SEQ ID NO:2 is intended that the amino acid sequence of the polypeptide is identical to the reference sequence except that the polypeptide sequence may include up to five amino acid alterations per each 100 amino acids of the reference amino acid of SEQ ID NO:2. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications, including but not limited to patents and patent applications, cited in this specification are herein incorporated by reference as if each individual publication were specifically and individually indicated to be incorporated by reference herein as though fully set forth.

SEQUENCE INFORMATION

SEQ ID NO:1
TTCTGCCAGA ATTCGGCTTA ACTCACTATA GGGCTCGAAC GGCCCGCCCG
GGCCAGGTCG AGAAGAAATC CCACTTGATT CAGATACTCT CCAAGATTGT
TAAAGGACTA ACTTCTCCCT CTTTATGCAC TTACGTCTGG AAGCATTCAG
AAAAGGAGGA ATGGAATAAC AAAGGTGACT CTGTAAGCAC ATTCTTCAAG
AAAGCCCAGG CACAGTCGAA CAGCGGTTTC CAGAAGCTGC TGATCCCATC
TTGCAAGGCT CAGCTCAACT CAGAGCCGCT GCTTCTTCCA AAGTGTACAT
CTTGGCGGAC CTTACGGAGG AAATCGGAAA GTAGAGACAC GTGAGGTCGT
GATGGAGCTC GAAGAAGACC TTAACGGAAG AGCAGACAAG AACTTCTCAA
AGATGGGCAA AAAGAGTAAA AAGGAGAAGA AAGAAAAGAA ACCAGCGGTC
AGTGTGCTCA CAATGTTTCG CTATGCAGGT TGGCTGGACA GATTTTACAT
GCTGCTGGGA ACTCTGGCGG CCATTATCCA TGGAATTGCG CTCCCACTTA
TGATGCTGGT CTTTGGAGAC ATGACAGATA GCTTTGCAAA TGTAGGAAAC
AACCGTAGTA TGAGTTTCTA CAATGCTACA GACATATATG CCAAGCTGGA
GGACGAAATG GCCACGTACG CCTACTATTA CACGGGCAAT GGTGCCGGTG
TGCTCATCGT TGCCTACATC CAGGTTTCAC TTTGGTGCCT GGCAGCTGGG
AGACAAATAC ACAAGATTAG GCAGAAGTTT TTCCATGCCA TCATGAATCA
GGAGATAGGC TGGTTTGACG TGCATGACGT TGGGGAGCTC AACACCCGGC
TCACAGATGA CGTCTCCAAA ATTAATGAAG GAATTGGTGA CAAAATTGGA
ATGTTCTTTC AGGCAATGGC AACATTTTTT GGTGGTTTTA TAATAGGATT
TACTCGCGGC TGGAAGCTAA CTCTTGTGAT TTTGGCCATC AGCCCTGTTC
TTGGACTGTC AGCTGGTATT TGGGCAAAGA TATTGTCTTC ATTTACTGAT
AAGGAACTCC AGGCTTATGC AAAAGCTGGA GCAGTTGCTG AAGAAGTCTT
AGCAGCCATC AGAACTGTGA TTGCCTTTGG AGGACAAAAG AAGGAACTTG
AAAGGTACAA TAACAATTTG GAAGAAGCTA AAAGGCTTGG GATAAAGAAA
GCTATCACGG CCAACATTTC CATGGGTGCA GCTTTTCTGC TTATCTATGC
ATCATATGCT CTGGCATTCT GGTATGGGAC TTCCTTGGTC ATCTCAAAAG
AATACACTAT TGGACAAGTG CTCACTGTCT TTTTTTCTGT ATTAATTGGA
GCATTCAGTG TTGGGCAGGC ATCTCCAAAT ATTGAAGCCT TCGCCAATGC
TAGAGGAGCA GCTTATGAAG TCTTCAGTAT AATTGATAAT AAGCCCAGTA
TAGACAGCTT CTCAAAGAGT GGGCACAAAC CCGACAACAT ACAAGGAAAT
TTGGAATTCA AAAATATTCA CTTCAGTTAC CCGTCTCGAA AAGACGTTCA
GATCTTGAAG GGCCTCAACC TGAAGGTGAA GAGCGGGCAG ACGGTAGCCC
TGGTTGGCAA CAGTGGCTGT GGGAAAAGCA CAACTGTCCA GCTGCTGCAG
AGGCTCTACG ACCCCATAGA GGGCGAGGTC AGTATCGACG GACAGGACAT
CAGGACCATC AATGTGAGGT ATCTGCGGGA AATCATTGGG GTGGTGAGTC
AGGAACCCGT GCTGTTTGCC ACCACAATTG CCGAAAACAT TCGCTATGGC
CGAGAAAACG TCACCATGGA TGAGATAGAG AAAGCTGTCA AGGAAGCCAA
TGCCTATGAT TTCATCATGA AACTGCCCCA CAAATTTGAC ACCCTGGTTG
GTGAGAGAGG GGCGCAGCTG AGTGGGGGAC AGAAACAGAG GATCGCCATT
GCCCGGGCCC TGGTCCGCAA CCCCAAGATC CTTTTGTTGG ATGAGGCCAC
GTCAGCCTTG GACACAGAAA GCGAAGCCGT GGTTCAGGCC GCTCTGGATA
AGGCTAGAGA AGGCCGGACC ACCATTGTGA TAGCTCACCG CTTGTCTACA
GTTCGCAATG CTGACGTCAT TGCTGGTTTT GATGGTGGTG TCATTGTGGA
GCAAGGAAAT CATGATGAGC TCATGAGAGA GAAAGGAATT TACTTCAAAC
TTGTCATGAC TCAGACAGCA GGAAATGAAA TTGAATTAGG AAATGAAGCT
TGTGAATCTA AAGACGGAAT TGATAATGTG GACATGTCTT CAAAAGATTC
GGGATCCAGT CTAATAAGAA GAAGATCAAC TCGCAAAAGC ATCCGTGGGC
CACATGATCA AGACGGGGAA CTTAGCACCA AAGAGGCTCT GGATGACGAC
GTACCTCCAG CTTCCTTTTG GCGGATCCTG AAGTTGAATT CAACTGAATG
GCCTTATTTT GTGGTTGGTG TATTTTGTGC CATAATAAAT GGAGGCTTGC
AACCAGCATT CTCCATAATA TTTTCAAAGG TTGTAGGGGT TTTTACAAAA
AATGACACCC CTGAAATCCA GCGGCAGAAC AGCAACTTGT TTTCTTTATT
GTTCCTGATC CTTGGGATCA TCTCTTTCAT TACGTTTTTC CTTCAGGGCT
TCACATTTGG CAAAGCTGGA GAGATCCTCA CCAAGCGACT CCGATACATG
GTCTTCAAAT CCATGCTGAG ACAGGACATA AGCTGGTTTG ATGACCCTAA
AAACACCACA GGAGCGCTGA CCACCAGGCT TGCCAATGAC GCTGCTCAAG
TGAAAGGGGC TACAGGGTCT AGGCTTGCTG TTATTACCCA GAACATAGCA
AATCTTGGGA CAGGCATCAT CATATCCCTG ATCTACGGCT GGCAATTGAC
ACTTTTACTC CTAGCAATTG TTCCCATCAT TGCTATAGCA GGAGTGGTTG
AAATGAAAAT GTTGTCTGGA CAAGCGCTGA AAGATAAGAA GGAACTAGAA
GGTTCTGGGA AGATCGCTAC AGAAGCAATT GAAAACTTTC GCACTGTCGT
CTCTTTGACT CGGGAGCAGA AGTTTGAAAC TATGTATGCC CAGAGCTTGC
AGATACCATA CAGAAATGCT TTGAAGAAAG CGCACGTCTT TGGGATCACT
TTCTCCTTCA CCCAGGCCAT GATGTATTTC TCCTATGCTG CTTGTTTCCG
GTTTGTAGCC TACTTGGTGG CACGAGAACT CATGACATTT GAAAATGTTC
TGTTAGTATT CTCAGCTATT GTCTTTGGTG CCATGGCAGT GGGGCAGGTC
AGTTCATTCG CTCCTGACTA CGCGAAAGCC AAAGTCTCGG CATCCCACAT
CATCAGGATC ATTGAGAAAA TCCCTGAGAT TGACAGCTAC AGCACGGAGG
GCTTGAAGCC TAATATGTTG GAAGGAAATG TGAAATTTAA TGGAGTCATG
TTCAACTATC CCACCCGACC CAACATCCCA GTGCTTCAGG GGCTGAGCCT
AGAGGTGAAG AAAGGGCAGA CGCTGGCCCT CGTGGGCAGC AGTGGCTGCG
GGAAGAGTAC AGTGGTCCAG CTGCTTGAGC GCTTCTATGA CCCCATGGCC
GGAACAGTGT TTCTAGATGG CAAAGAAATA AAGCAACTCA ATGTCCAGTG
GCTCCGCGCC CACCTGGGCA TTGTGTCCCA GGAGCCCATC CTGTTTGACT
GCAGCATCGC CGAGAACATT GCCTACGGAG ACAACAGCCG TGTCGTGTCT
CATAAGGAGA TCGTGAAGGC AGCCAAGGAG GCCAACATCC ACCAGTTCAT
CGACTCACTG CCTGAGAAAT ACAACACCAG AGTGGGAGAC AAAGGGACTC
AGCTGTCGGG CGGGCAGAAG CAGCGCATCG CCATCGCGCG CGCCCTCGTC
AGACAGCCTC ACATCTTACT TCTGGATGAA GCGACATCAG CTCTGGATAC
GGAGAGTGAA AAGGTCGTCC AGGAAGCGCT GGACAAAGCC AGGGAAGGCC
GCACCTGCAT TGTGATCGCG CACCGCCTGT CCACCATCCA GAACGCAGAC
TTGATCGTGG TGATTCAGAA CGGCCAGGTC AAGGAGCACG GCACCCACCA
GCAGCTGCTG GCCCAGAAAG GCATCTATTT CTCGATGGTC AGTGTGCAGG
CTGGAGCAAA GCGCTCATGA ACTGTGACCA TGCGAGATGT TAAATATTTT
TAATGTTTGT ATTAATATAT GACACTTAAT CAAAGTCAAA AGGAAAACAC
TTACTAGAAT AGTCAGTTAT CTATTTCCTG TCACAAAGGA AAGCATTTAG
TCCATTTTAG AGTCTTCAGA GACTTTGTAA TTAAAAGAAC AAAAATAGAT
ACATCATCAA ATGGAATTC
SEQ ID NO:2
MELEEDLNGR ADKNFSKMGK KSKKEKKEKK PAVSVLTMFR YAGWLDRFYM
LLGTLAAIIH GIALPLMMLV FGDMTDSFAN VGNNRSMSFY NATDIYAKLE
DEMATYAYYY TGNGAGVLIV AYIQVSLWCL AAGRQIHKIR QKFFHAIMNQ
EIGWFDVHDV GELNTRLTDD VSKINEGIGD KIGMFFQAMA TFFGGFIIGF
TRGWKLTLVI LAISPVLGLS AGIWAKILSS FTDKELQAYA KAGAVAEEVL
AAIRTVIAFG GQKKELERYN NNLEEAKRLG IKKAITANIS MGAAFLLIYA
SYALAFWYGT SLVISKEYTI GQVLTVFFSV LIGAFSVGQA SPNIEAFANA
RGAAYEVFSI IDNKPSIDSF SKSGHKPDNI QGNLEFKNIH FSYPSRKDVQ
ILKGLNLKVK SGQTVALVGN SGCGKSTTVQ LLQRLYDPIE GEVSIDGQDI
RTINVRYLRE IIGVVSQEPV LFATTIAENI RYGRENVTMD EIEKAVKEAN
AYDFIMKLPH KFDTLVGERG AQLSGGQKQR IAIARALVRN PKILLLDEAT
SALDTESEAV VQAALDKARE GRTTIVIAHR LSTVRNADVI AGFDGGVIVE
QGNHDELMRE KGIYFKLVMT QTAGNEIELG NEACESKDGI DNVDMSSKDS
GSSLIRRRST RKSIRGPHDQ DGELSTKEAL DDDVPPASFW RILKLNSTEW
PYFVVGVFCA IINGGLQPAF SIIFSKVVGV FTKNDTPEIQ RQNSNLFSLL
FLILGIISFI TFFLQGFTFG KAGEILTKRL RYMVFKSMLR QDISWFDDPK
NTTGALTTRL ANDAAQVKGA TGSRLAVITQ NIANLGTGII ISLIYGWQLT
LLLLAIVPII AIAGVVEMKM LSGQALKDKK ELEGSGKIAT EAIENFRTVV
SLTREQKFET MYAQSLQIPY RNALKKAHVF GITFSFTQAM MYFSYAACFR
FDAYLVAREL MTFENVLLVF SAIVFGAMAV GQVSSFAPDY AKAKVSASHI
IRIIEKIPEI DSYSTEGLKP NMLEGNVKFN GVMFNYPTRP NIPVLQGLSL
EVKKGQTLAL VGSSGCGKST VVQLLERFYD PMAGTVFLDG KEIKQLNVQW
LRAHLGIVSQ EPILFDCSIA ENIAYGDNSR VVSHKEIVKA AKEANIHQFI
DSLPEKYNTR VGDKGTQLSG GQKQRIAIAR ALVRQPHILL LDEATSALDT
ESEKVVQEAL DKAREGRTCI VIAHRLSTIQ NADLIVVIQN GQVKEHGTHQ
QLLAQKGIYF SMVSVQAGAK RS*
SEQ ID NO:3
TTCTGCCAGAATTCGGCTTAACTCACTATAGGGCTCGAACGGCCCGCCCGGGCCAGGTCG
AGAAGAAATCCCACTTGATTCAGATACTCTCCAAGATTGTTAAAGGACTAACTTCTCCCT
CTTTATGCACTTACGTCTGGAAGCATTCAGAAAAGGAGGAATGGAATAACAAAGGTGACT
CTGTAAGCACATTCTTCAAGAAAGCCCAGGCACAGTCGAACAGCGGTTTCCAGAAGCTGC
TGATCCCATCTTGCAAGGCTCAGCTCAACTCAGAGCCGCTGCTTCTTCCAAAGTGTACAT
CTTGGCGGACCTTACGGAGGAAATCGGAAAGTAGAGACACGTGAGGTCGTGATGGAGCTC
GAAGAAGACCTTAACGGAAGAGCAGACAAGAACTTCTCAAAGATGGGCAAAAAGAGTAAA
AAGGAGAAGAAAGAAAAGAAACCAGCGGTCAGTGTGCTCACAATGTTTCGCTATGCAGGT
TGGCTGGACAGATTTTACATGCTGCTGGGAACTCTGGCGGCCATTATCCATGGAATTGCG
CTCCCACTTATGATGCTGGTCTTTGGAGACATGACAGATAGCTTTGCAAATGTAGGAAAC
AACCGTAGTATGAGTTTCTACAATGCTACAGACATATATGCCAAGCTGGAGGACGAAATG
ACCACGTACGCCTACTATTACACGGGCATTGGTGCCGGTGTGCTCATCGTTGCCTACATC
CAGGTTTCCACTTGGTGCCTGGCAGCTGGGAGACAAATACACAAGATTAGGCAGAAGTTT
TTCCATGCCATCATGAATCAGGAGATAGGCTGGTTTGACGTGCATGACGTTGGGGAGCTC
AACACCCGGCTCACAGATGACGTCTCCAAAATTAATGAAGGAATTGGTGACAAAATTGGA
ATGTTCTTTCAGGCAATGGCAACATTTTTTGGTGGTTTTATAATAGGATTTACTCGCGGC
TGGAAGCTAACTCTTGTGATTTTGGCCATCAGCCCTGTTCTTGGACTGTCAGCTGGTATT
TGGGCAAAGATATTGTCTTCATTTACTGATAAGGAACTCCAGGCTTATGCAAAAGCTGGA
GCAGTTGCTGAAGAAGTCTTAGCAGCCATCAGAACTGTGATTGCCTTTGGAGGACAAAAG
AAGGAACTTGAAAGGTACAATAACAATTTGGAAGAAGCTAAAAGGCTTGGGATAAAGAAA
GCTATCACGGCCAACATTTCCATGGGTGCAGCTTTTCTGCTTATCTATGCATCATATGCT
CTGGCATTCTGGTATGGGACTTCCTTGGTCATCTCAAAAGAATACACTATTGGACAAGTG
CTCACTGTCTTTTTTTCTGTATTAATTGGAGCATTCAGTGTTGGGCAGGCATCTCCAAAT
ATTGAAGCCTTCGCCAATGCTAGAGGAGCAGCTTATGAAGTCTTCAGTATAATTGATAAT
AAGCCCAGTATAGACAGCTTCTCAAAGAGTGGGCACAAACCCGACAACATACAAGGAAAT
TTGGAATTCAAAAATATTCACTTCAGTTACCCGTCTCGAAAAGACGTTCAGATCTTGAAG
GGCCTCAACCTGAAGGTGAAGAGCGGGCAGACGGTAGCCCTGGTTGGCAACAGTGGCTGT
GGGAAAAGCACAACTGTCCAGCTGCTGCAGAGGCTCTACGACCCCATAGAGGGCGAGGTC
AGTATCGACGGACAGGACATCAGGACCATCAATGTGAGGTATCTGCGGGAAATCATTGGG
GTGGTGAGTCAGGAACCCGTGCTGTTTGCCACCACAATTGCCGAAAACATTCGCTATGGC
CGAGAAAACGTCACCATGGATGAGATAGAGAAAGCTGTCAAGGAAGCCAATGCCTATGAT
TTCATCATGAAACTGCCCCACAAATTTGACACCCTGGTTGGTGAGAGAGGGGCGCAGCTG
AGTGGGGGACAGAAACAGAGGATCGCCATTGCCCGGGCCCTGGTCCGCAACCCCAAGATC
CTTTTGTTGGATGAGGCCACGTCAGCCTTGGACACAGAAAGCGAAGCCGTGGTTCAGGCC
GCTCTGGATAAGGCTAGAGAAGGCCGGACCACCATTGTGATAGCTCACCGCTTGTCTACA
GTTCGCAATGCTGAYGTCATTGCTGGTTTTGATGGTGGTGTCATTGTGGAGCAAGGAAAT
CATGATGAGCTCATGAGAGAGAAAGGAATTTACTTCAAACTTGTCATGACTCAGACAGCA
GGAAATGAAATTGAATTAGGAAATGAAGCTTGTGAATCTAAAGAYGGAATTGATAATGTG
GACATGTCTTCAAAAGATTCRGGATCCAGTCTAATAAGAAGAAGATCAACTCGCAAAAGC
ATCCGTGGGCCACATGATCAAGACGGGGAACTTAGCACCAAAGAGGCTCTGGATGACGAC
GTACCTCCAGCTTCCTTTTGGCGGATCCTGAAGTTGAATTCAACTGAATGGCCTTATTTT
GTGGTTGGTGTATTTTGTGCCATAATAAATGGAGGCTTGCAACCAGCATTCTCCATAATA
TTTTCAAAGGTTGTAGGGGTTTTTACAAAAAATGACACCCCTGAAATCCAGCGGCAGAAC
AGCAACTTGTTTTCTTTATTGTTCCTGATCCTTGGGATCATCTCTTTCATTACGTTTTTC
CTTCAGGGCTTCACATTTGGCAAAGCTGGAGAGATCCTCACCAAGCGACTCCGATACATG
GTCTTCAAATCCATGCTGAGACAGGACATAAGCTGGTTTGATGACCCTAAAAACACCACA
GGAGCGCTGACCACCAGGCTTGCCAATGACGCTGCTCAAGTGAAAGGGGCTACAGGGTCT
AGGCTTGCTGTTATTACCCAGAACATAGCAAATCTTGGGACAGGCATCATCATATCCCTG
ATCTACGGCTGGCAATTGACACTTTTACTCCTAGCAATTGTTCCCATCATTGCTATAGCA
GGAGTGGTTGAAATGAAAATGTTGTCTGGACAAGCGCTGAAAGATAAGAAGGAACTAGAA
GGTTCTGGGAAGATCGCTACAGAAGCAATTGAAAACTTTCGCACTGTCGTCTCTTTGACT
CGGGAGCAGAAGTTTGAAACTATGTATGCCCAGAGCTTGCAGATACCATACAGAAATGCT
TTGAAGAAAGCGCACGTCTTTGGGATCACTTTCTCCTTCACCCAGGCCATGATGTATTTC
TCCTATGCTGCTTGTTTCCGGTTTGATGCCTACTTGGTGGCACGAGAACTCATGACATTT
GAAAATGTTCTGTTAGTATTCTCAGCTATTGTCTTTGGTGCCATGGCAGTGGGGCAGGTC
AGTTCATTCGCTCCTGACTACGCGAAAGCCAAAGTCTCGGCATCCCACATCATCAGGATC
ATTGAGAAAATCCCTGAGATTGACAGCTACAGCACGGAGGGCTTGAAGCCTAATATGTTG
GAAGGAAATGTGAAATTTAATGGAGTCATGTTCAACTATCCCACCCGACCCAACATCCCA
GTGCTTCAGGGGCTGAGCCTAGAGGTGAAGAAAGGGCAGACGCTGGCCCTCGTGGGCAGC
AGTGGCTGCGGGAAGAGTACAGTGGTCCAGCTGCTTGAGCGCTTCTATGACCCCATGGCC
GGAACAGTGTTTCTAGATGGCAAAGAAATAAAGCAACTCAATGTCCAGTGGCTCCGCGCC
CACCTGGGCATTGTGTCCCAGGAGCCCATCCTGTTTGACTGCAGCATCGCCGAGAACATT
GCCTACGGAGACAACAGCCGTGTCGTGTCTCATAAGGAGATCGTGAAGGCAGCCAAGGAG
GCCAACATCCACCAGTTCATCGACTCACTGCCTGAGAAATACAACACCAGAGTGGGAGAC
AAAGGGACTCAGCTGTCGGGCGGGCAGAAGCAGCGCATCGCCATCGCGCGCGCCCTCGTC
AGACAGCCTCACATCTTACTTCTGGATGAAGCGACATCAGCTCTGGATACGGAGAGTGAA
AAGGTCGTCCAGGAAGCGCTGGACAAAGCCAGGGAAGGCCGCACCTGCATTGTGATCGCG
CACCGCCTGTCCACCATCCAGAACGCAGACTTGATCGTGGTGATTCAGAACGGCCAGGTC
AAGGAGCACGGCACCCACCAGCAGCTGCTGGCCCAGAAAGGCATCTATTTCTCGATGGTC
AGTGTGCAGGCTGGAGCAAAGCGCTCATGAACTGTGACCATGCGAGATGTTAAATATTTT
TAATGTTTGTATTAATATATGACACTTAATCAAAGTCAAAAGGAAAACACTTACTAGAAT
AGTCAGTTATCTATTTCCTGTCACAAAGGAAAGCATTTAGTCCATTTTAGAGTCTTCAGA
GACTTTGTAATTAAAAGAACAAAAATAGATACATCATCAAATGGAATTCCTGCAGCCCGG
GGGATCCACTAGTTCTAGAGCGGCCGCCACCGCGGTGGAGCTCCA
SEQ ID NO:4
MELEEDLNGRADKNFSKMGKKSKKEKKEKKPAVSVLTMFRYAGWLDRFYMLLGTLAAIIH
GIALPLMMLVFGDMTDSFANVGNNRSMSFYNATDIYAKLEDEMTTYAYYYTGIGAGVLIV
AYIQVSTWCLAAGRQIHKIRQKFFHAIMNQEIGWFDVHDVGELNTRLTDDVSKINEGIGD
KIGMFFQAMATFFGGFIIGFTRGWKLTLVILAISPVLGLSAGIWAKILSSFTDKELQAYA
KAGAVAEEVLAAIRTVIAFGGQKKELERYNNNLEEAKRLGIKKAITANISMGAAFLLIYA
SYALAFWYGTSLVISKEYTIGQVLTVFFSVLIGAFSVGQASPNIEAFANARGAAYEVFSI
IDNKPSIDSFSKSGHKPDNIQGNLEFKNIHFSYPSRKDVQILKGLNLKVKSGQTVALVGN
SGCGKSTTVQLLQRLYDPIEGEVSIDGQDIRTINVRYLREIIGVVSQEPVLFATTIAENI
RYGRENVTMDEIEKAVKEANAYDFTMKLPNKFDTLVGERGAQLSGGQKQRIAIARALVRN
PKILLLDEATSALDTESEAVVQAALDKAREGRTTIVIAHRLSTVRNADVIAGFDGGVIVE
QGNHDELMREKGIYFKLVMTQTAGNEIELGNEACESKDGIDNVDMSSKDSGSSLIRRRST
RKSIRGPHDQDGELSTKEALDDDVPPASFWRILKLNSTEWPYFVVGVFCAIINGGLQPAF
SIIFSKVVGVFTKNDTPEIQRQNSNLFSLLFLILGIISFITFFLQGFTFGKAGEILTKRL
RYMVFKSMLRQDISWFDDPKNTTGALTTRLANDAAQVKGATGSRLAVITQNIANLGTGII
ISLIYGWQLTLLLLAIVPIIAIAGVVEMKMLSGQALKDKKELEGSGKIATEAIENFRTVV
SLTREQKFETMYAQSLQIPYRNALKKAHVFGITFSFTQAMMYFSYAACFRFDAYLVAREL
MTFENVLLVFSAIVFGAMAVGQVSSFAPDYAKAKVSASHIIRIIEKIPEIDSYSTEGLKP
NMLEGNVKFNGVMFNYPTRPNIPVLQGLSLEVKKGQTLALVGSSGCGKSTVVQLLERFYD
PMAGTVFLDGKEIKQLNVQWLRAHLGIVSQEPILFDCSIAENIAYGDNSRVVSHKEIVKA
AKEANIEQFIDSLPEKYNTRVGDKGTQLSGGQKQRIAIARALVRQPHILLLDEATSALDT
ESEKVVQEALDKAREGRTCIVIAHRLSTIQNADLIVVIQNGQVKEHGTHQQLLAQKGIYF
SMVSVQAGAKRS*

What is claimed is:

1. An isolated polypeptide selected from the group consisting of:
(i) an isolated polypeptide having at least a 95% identity to the amino acid sequence of SEQ ID NO:2 over the entire length of SEQ ID NO:2;
(ii) an isolated polypeptide comprising the amino acid sequence of SEQ ID NO:2 or
(iii) an isolated polypeptide which is the amino acid sequence of SEQ ID NO:2.

2. An isolated polynucleotide selected from the group consisting of:
(i) an isolated polynucleotide comprising a nucleotide sequence encoding a polypeptide that has at least a 95% identity to the amino acid sequence of SEQ ID NO:2, over the entire length of SEQ ID NO:2;
(ii) an isolated polynucleotide comprising a nucleotide sequence that has at least a 95% identity over its entire length to a nucleotide sequence encoding the polypeptide of SEQ ID NO: 2;
(iii) an isolated polynucleotide comprising a nucleotide sequence which has at least a 95% identity to that of SEQ ID NO:1 over the entire length of SEQ ID NO:1;
(iv) an isolated polynucleotide comprising a nucleotide sequence encoding the polypeptide of SEQ ID NO:2;
(vi) an isolated polynucleotide which is the polynucleotide of SEQ ID NO:1; or
(vi) an isolated polynucleotide obtainable by screening an appropriate library under stringent hybridization conditions with a labeled probe having the sequence of SEQ ID NO:1 or a fragment thereof.;
or a nucleotide sequence complementary to said isolated polynucleotide.

3. A method for screening to identify compounds which stimulate or which inhibit the function of the polypeptide of claim 1, which comprises a method selected from the group consisting of:
(a) measuring the binding of a candidate compound to the polypeptide (or to the cells or membranes bearing the polypeptide) or a fusion protein thereof by means of a label directly or indirectly associated with the candidate compound;
(b) measuring the binding of a candidate compound to the polypeptide (or to the cells or membranes bearing the polypeptide) or a fusion protein thereof in the presense of a labeled competitior;
(c) testing whether the candidate compound results in a signal generated by activation or inhibition of the polypeptide, using detection systems appropriate to the cells or cell membranes bearing the polypeptide;
(d) mixing a candidate compound with a solution containing a polypeptide of claim 1, to form a mixture, measuring activity of the polypeptide in the mixture, and comparing the activity of the mixture to a standard; or
(e) detecting the effect of a candidate compound on the production of mRNA encoding said polypeptide and said polypeptide in cells.

4. An agonist or an antagonist of the polypeptide of claim 1.

5. A method for screening to identify compounds which neither stimulate nor which inhibit the function of the polypeptide of claim 1, which comprises a method selected from the group consisting of:
(a) measuring the binding of a candidate compound to the polypeptide (or to the cells or membranes bearing the polypeptide) or a fusion protein thereof by means of a label directly or indirectly associated with the candidate compound;
(b) measuring the binding of a candidate compound to the polypeptide (or to the cells or membranes bearing the polypeptide) or a fusion protein thereof in the presense of a labeled competitior;
(c) testing whether the candidate compound results in a signal generated by activation or inhibition of the polypeptide, using detection systems appropriate to the cells or cell membranes bearing the polypeptide;
(d) mixing a candidate compound with a solution containing a polypeptide of claim 1, to form a mixture, measuring activity of the polypeptide in the mixture, and comparing the activity of the mixture to a standard; or
(e) detecting the effect of a candidate compound on the production of mRNA encoding said polypeptide and said polypeptide in cells.

6. An expression system comprising a polynucleotide capable of producing a polypeptide of claim 1 when said expression system is present in a compatible host cell.

7. A process for producing a recombinant host cell comprising transforming or transfecting a cell with the expression system of claim 8 such the the host cell, under appropriate culture conditions, produces a polypeptide comprising an amino acid sequence having at least a 95% identity to the amino acid sequence of SEQ ID NO:2 over the entire length of SEQ ID NO:2.

8. A recombinant host cell produced by the process of claim 7.

9. A membrane of a recombinant host cell of claim 8 expressing a polypeptide comprising an amino acid sequence having at least a 95% identity to the amino acid sequence of SEQ ID NO:2 over the entire length of SEQ ID NO:2.

10. A process for producing a polypeptide comprising culturing a host cell of claim 9 under conditions sufficient for the production of said polypeptide and recovering the polypeptide from the culture.

11. An isolated polynucleotide selected form the group consisting of:
(a) an isolated polynucleotide comprising a nucleotide sequence which has at least 95% identity to SEQ ID NO:3 over the entire length of SEQ ID NO:3;
(b) an isolated polynucleotide comprising a nucleotide sequence which has at least 95% identity to SEQ ID NO:1
over the entire length of SEQ ID NO:3;
(e) an isolated polynucleotide comprising the polynucleotide of SEQ ID NO:3;
(d) the polynucleotide of SEQ ID NO:3; or
(e) an isolated polynucleotide comprising a nucleotide sequence encoding a polypeptide which has at least 95% identity to the amino acid sequence of SEQ ID NO:4, over the entire length of SEQ ID NO: 4.

12. A polypeptide selected from the group consisting of:
(a) a polypeptide which comprises an amino acid sequence which has at least 95% identity to that of SEQ ID NO:4 over the entire length of SEQ ID NO:4;
(b) a polypeptide in which the amino acid sequence has at least 95% identity to the amino acid sequence of SEQ ID NO:4 over the entire length of SEQ ID NO:4;
(c) a polypeptide which comprises the amino acid of SEQ ID NO:4;
(d) a polypeptide which is the polypeptide of SEQ ID NO:4;
(e) a polypeptide which is encoded by a polynucleotide comprising the sequence contained in SEQ ID NO:3.
